(19) United States
(12) Patent Application Publication
(10) Pub. No.: US 2024/0221873 A1
(43) Pub. Date: Jul. 4, 2024

(54) SYSTEMS AND METHODS FOR EVALUATING PROGRAMS

(71) Applicant: UnitedHealth Group Incorporated, Minnetonka, MN (US)

(72) Inventors: Changchun Alan WANG, Anoka, MN (US); Andrea MATHIS, Fulshear, TX (US); Wenjun HU, North Potomac, MD (US)

(73) Assignee: UnitedHealth Group Incorporated, Minnetonka, MN (US)

(21) Appl. No.: 18/149,294

(22) Filed: Jan. 3, 2023

(51) Int. Cl. G16H 10/20

(52) U.S. Cl. CPC G16H 10/20

(57) ABSTRACT

Systems and computer-implemented method for evaluating programs are disclosed. A computer-implemented method includes determining a propensity score, using a propensity score model, for each patient among multiple patients. The multiple patients include treatment patients and control patients, and the propensity score represents a probability of assignment to a treatment group. The method includes assigning a random value to each patient in an assignment group. The assignment group includes at least one of the treatment patients or the control patients. The method includes sorting the patients based on the assigned random values and matching, based on the sorted patients and the determined propensity scores, each treatment patient to a control patient to create multiple matches. Each match includes one treatment patient and at least one control patient. The method includes performing, based on the multiple matches, one or more actions related to the multiple patients.

### TECHNICAL FIELD

Various embodiments of the present disclosure relate generally to systems and methods for evaluating programs, and more particularly to, systems, computer-implemented methods, and non-transitory computer readable mediums for estimating savings in an intervention or other program using propensity score matching.

### BACKGROUND

Propensity score matching (PSM) without replacement is a quasi-experimental method that can be used to evaluate a clinic or treatment program. In PSM, observations in an experimental group are matched to a closest component in a control group. Once a first observation is matched to its closest component, a second observation is matched among the remaining components in the control group. However, because two or more observations may both be closest to a same control group component, the order of data in which matching occurs has a significant impact on results. Thus, a result that derives from a comparison of each observation and its paired control group component could be atypical, misleading, or incorrect.

In the context of using PSM for savings estimates, which may have positive and negative values indicating money saved or lost, a savings estimate from PSM might be misleading in two ways. First, the savings estimate may have an opposite or reverse sign or direction of its true sign; for example, an actual positive savings may be reported as a negative savings or vice versa. Second, even if a sign of the savings is correctly identified as positive or negative, its reported value or magnitude may still be significantly off from a true or real savings value. Therefore, a need exists for a system and method for more accurate PSM results that are not misleading.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, computer-implemented methods, systems, and non-transitory computer readable mediums are disclosed for evaluating programs.

In one aspect, a computer-implemented method for evaluating programs is disclosed. The method may include determining a propensity score, using a propensity score model, for each patient among multiple patients. The multiple patients may include a plurality of treatment patients and a plurality of control patients, and the propensity score may represent a probability of assignment to a treatment group. The method may further include assigning a random value to each patient in an assignment group. The assignment group may include at least one of the plurality of treatment patients or the plurality of control patients. The method may further include sorting the plurality of patients based on the assigned random values and matching, based on the sorted patients and the determined propensity scores, each treatment patient to a control patient to create multiple matches. Each match may include one treatment patient and at least one control patient. The method may further include performing, based on the multiple matches, one or more actions related to the multiple patients.

The method may further include repeating assigning the random value, sorting the plurality of patients, and matching, wherein, upon repeating assigning the random value, each patient in the assignment group is assigned a random value that is different from the random value previously assigned.

Assigning the random value may be based on a first seed. The method may further include repeating assigning the random value based on a second seed.

Sorting may be based on the determined propensity scores. Matching may be performed in order of the sorted patients.

The method may further include removing one or more outlier matches from the multiple matches to create a subgroup of matches. Performing the one or more actions may be based on the subgroup of matches.

Each patient may be associated with a value. Removing of the one or more outlier matches may include detecting one or more patients having an associated value within a highest and/or lowest percentage among all associated values of the multiple patients, and excluding the matches among the multiple matches that include any of the detected patients.

The multiple patients may be associated with one or more categories.

Performing the one or more actions may be based on the one or more categories.

The method may further include receiving a plurality of values. Each value associated with each patient among the multiple patients may be based on a cost for treatment.

Performing of the one or more actions may further include determining a distribution of the values, and displaying, on a user interface, the determined distribution.

Determining the distribution of the values may include comparing, within each of the multiple matches, the value associated with the treatment patient and the value associated with the control patient. Assigning, sorting, matching, and performing may be repeated a plurality of iterations to determine a plurality of distributions of the values, each distribution corresponding to an iteration. The method may further comprise determining, based on the plurality of distributions, a representative iteration among the plurality of iterations, and displaying, on the user interface, a table indicating the representative iteration. Determining the representative iteration is based on a number of the values of the distribution for each iteration that have a same sign as a mean of the distribution and a magnitude of a difference between the values of the distribution for each iteration and the mean.

The method may further include determining a centroid based on the magnitudes and signs of the values for all iterations. Determining the representative iteration may be based on a difference between each iteration and the determined centroid.

The matching may be based on one or more characteristics of the multiple patients.

In another aspect, a system for evaluating programs is disclosed. The system may include a memory having processor-readable instructions stored therein and a processor configured to access the memory and execute the processor-readable instructions to perform operations. The operations may include determining a propensity score, using a propensity score model, for each patient among multiple patients. The multiple patients may include a plurality of treatment patients and a plurality of control patients. The propensity score may represent a probability of assignment to treatment. The operations may further include assigning a random value to each patient in an assignment group. The assignment group may include at least one of the plurality of treatment patients or the plurality of control patients. The operations may further include sorting the plurality of patients based on the assigned random values, matching, based on the sorted patients and the determined propensity scores, each treatment patient to a control patient to create multiple matches, each match including one treatment patient and at least one control patient, and performing, based on the multiple matches, one or more actions related to the multiple patients.

The operations may further include repeating assigning the random value, sorting the plurality of patients, and matching. Upon repeating assigning the random value, each patient in the assignment group may be assigned a random value that is different from the random value previously assigned.

The assigning, sorting, matching, and performing is repeated a plurality of iterations to determine a plurality of distributions of the values. Each distribution may correspond to an iteration. The operations may further include determining, based on the plurality of distributions, a representative iteration among the plurality of iterations, and displaying, on the user interface, a table indicating the representative iteration.

In yet another aspect, a non-transitory computer-readable medium storing a set of instructions that, when executed by a processor, perform operations for evaluating programs is disclosed. The operations may include determining a propensity score, using a propensity score model, for each patient among multiple patients. The multiple patients may include a plurality of treatment patients and a plurality of control patients. The propensity score may represent a probability of assignment to treatment. The operations may further include assigning a random value to each patient in an assignment group. The assignment group may include at least one of the plurality of treatment patients or the plurality of control patients. The operations may further include sorting the plurality of patients based on the assigned random values, matching, based on the sorted patients and the determined propensity scores, each treatment patient to a control patient to create multiple matches, each match including one treatment patient and at least one control patient, and performing, based on the multiple matches, one or more actions related to the multiple patients.

The operations may further comprise repeating assigning the random value, sorting the plurality of patients, and matching. Upon repeating assigning the random value, each patient may be assigned a random value that is different from the random value previously assigned.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 depicts a block diagram of an exemplary system for evaluating a an intervention or case management program, according to one or more embodiments.

FIG. 2 depicts a flowchart illustrating an exemplary method for performing one or more actions using propensity score matching, according to one or more embodiments.

FIG. 3A depicts an exemplary match in a first simulation or iteration of the method of FIG. 2, and FIG. 3B depicts an exemplary match in a second simulation or iteration of the method of FIG. 2 where some, but not all, patients may be re-sorted, according to one or more embodiments. FIG. 3C depicts an exemplary match in a first simulation of the method of FIG. 2, and FIG. 3D depicts an exemplary match in a second simulation or iteration of the method of FIG. 2 where multiple groups of patients may be re-sorted, according to one or more embodiments. FIG. 3E depicts an exemplary match in a first simulation of the method of FIG. 2, and FIG. 3D depicts an exemplary match in a second simulation or iteration of the method of FIG. 2 where all patients may be re-sorted, according to one or more embodiments.

FIG. 4 depicts an exemplary analysis or output based on all simulations or iterations of the method of FIG. 2, according to one or more embodiments.

FIG. 5A depicts a flowchart illustrating an exemplary method for determining a typical scenario among all simulations, and FIG. 5B depicts an exemplary analysis or output of the determined typical scenario, according to one or more embodiments.

FIG. 6 depicts an implementation of a computer system that may execute techniques presented herein.

### DETAILED DESCRIPTION

Various embodiments of the present disclosure relate generally to evaluating interventions and/or clinic programs. More particularly, various embodiments of the present disclosure relate to systems, computer-implemented methods, and non-transitory computer readable mediums for evaluating an intervention (e.g., case management, disease management, or population health management program) using propensity score matching.

Running hundreds of interventions or programs could cost tens of millions dollars per year. As discussed above, randomly reporting a scenario or result based on a PSM analysis to evaluate savings in an intervention may produce unreliable, inaccurate, or misleading results. Therefore, the embodiments of the present disclosure are directed to solving, mitigating, or rectifying the above-mentioned issues. Aspects disclosed herein may evaluate interventions and/or other programs or studies in a more comprehensive way and provide more confidence for clinicians, operation, and senior management to make more accurate or more informed business decisions. Aspects disclosed herein may have a significant impact for intervention evaluation involving matching or random selection of control group in the health industry. Aspects disclosed herein may reduce or minimize the significance of data order when running PSM. For example, aspects disclosed herein may run PSM on a same set of data multiple times using a different order of data and generate different matches and/or results each time. Aspects disclosed herein may increase an amount of data obtained from propensity score matches and may increase accuracy in the data obtained. In addition, aspects disclosed herein may find a savings estimate distribution and find a typical scenario to present data. Aspects disclosed herein may provide multiple rounds of analysis. Aspects disclosed herein may require an exact or almost exact match for certain predictors. Aspects disclosed herein may include more factors that impact final matching results. Aspects disclosed herein may order at least some data based on PSM score before matching. Aspects disclosed herein may allow for large sample sizes or large data sets for matching. If the sample size for matching is large, aspects disclosed herein may use high-performance distributed computing for a simulation process, for example, using multi-threaded and/or in-memory run systems or processes.

The terminology used herein may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” “includes,” “including,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The term “clinician” may include, for example, without limitation, any person, organization, and/or collection of persons that provides medical care (i.e., health care provider). For example, a clinician may include a physician, a nurse, a psychologist, an optometrist, a veterinarian, a physiotherapist, a dentist, and a physician assistant.

As used herein, a “machine-learning model” generally encompasses instructions, data, and/or a model configured to receive input, and apply one or more of a weight, bias, classification, or analysis on the input to generate an output. The output may include, for example, an analysis based on the input, a prediction, suggestion, or recommendation associated with the input, a dynamic action performed by a system, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as k-nearest neighbors, linear regression, logistical regression, random forest, gradient boosted machine (GBM), support-vector machine, deep learning, text classifiers, image recognition classifiers, You Only Look Once (YOLO), a deep neural network, greedy matching, propensity score matching, and/or any other suitable machine-learning technique that solves problems specifically addressed in the current disclosure. Supervised, semi-supervised, and/or unsupervised training may be employed. For example, supervised learning may include providing training data and labels corresponding to the training data, e.g., as ground truth. Unsupervised approaches may include clustering, classification, principal component analysis (PCA) or the like. K-means clustering or K-Nearest Neighbors may also be used, which may be supervised or unsupervised.

Combinations of K-Nearest Neighbors and an unsupervised cluster technique may also be used. Other models for detecting objects in contents/files, such as documents, images, pictures, drawings, and media files may be used as well. Any suitable type of training may be used, e.g., stochastic, gradient boosted, random seeded, recursive, epoch or batch-based, etc.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

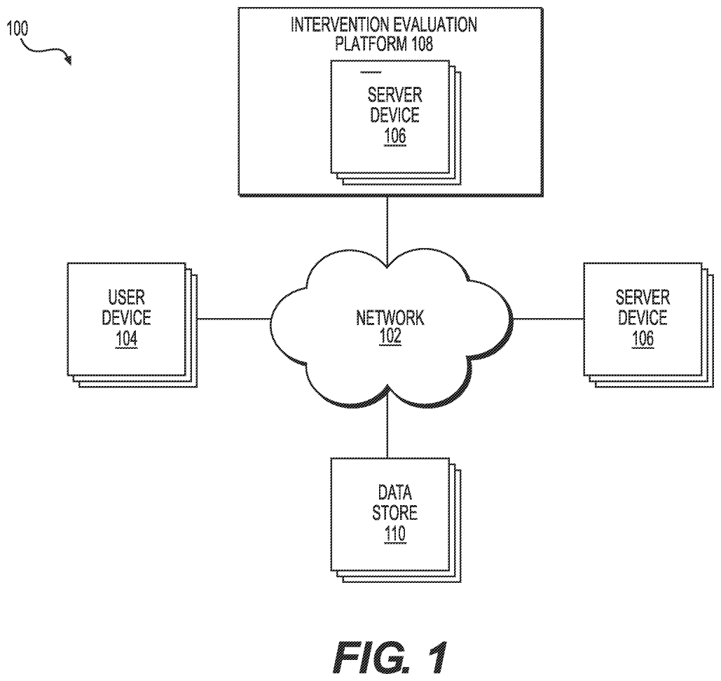

Referring now to the appended drawings, FIG. 1 depicts a block diagram of an exemplary system 100 for evaluating an intervention (e.g., case management, disease management, or population health management program), according to one or more embodiments. As illustrated in FIG. 1, the system 100 may include a network 102, one or more user devices 104, one or more server devices 106, an intervention evaluation platform 108, which may include one or more of the server devices 106, and one or more data stores 110.

The network 102 may include a wired and/or wireless network that may couple devices so that communications can be exchanged, such as between a server and a user device or other types of devices, including between wireless devices coupled via a wireless network, for example. A network can also include mass storage, such as network attached storage (NAS), a storage area network (SAN), or other forms of computer or machine-readable media, for example. A network can include the Internet, one or more local area networks (LANs), one or more wide area networks (WANs), wire-line type connections, wireless type connections, cellular or any combination thereof. Likewise, sub-networks, which can employ differing architectures or can be compliant or compatible with differing protocols, can interoperate within a larger network. Various types of devices can, for example, be made available to provide an interoperable capability for differing architectures or protocols. As one illustrative example, a router can provide a link between otherwise separate and independent LANs.

Furthermore, devices or user devices, such as computing devices or other related electronic devices can be remotely coupled to a network, such as via a wired or wireless line or link, for example.

In certain non-limiting embodiments, a “wireless network” should be understood to couple user devices with a network. A wireless network can include virtually any type of wireless communication mechanism by which signals can be communicated between devices, between or within a network, or the like. A wireless network can employ standalone ad-hoc networks, mesh networks, wireless land area network (WLAN), cellular networks, or the like. A wireless network may be configured to include a system of terminals, gateways, routers, or the like coupled by wireless radio links, or the like, which can move freely, randomly, or organize themselves arbitrarily, such that network topology can change, at times even rapidly.

A wireless network can further employ a plurality of network access technologies, including Wi-Fi, Long Term Evolution (LTE), WLAN, Wireless Router (WR) mesh, or 2nd, 3rd, 4th, 5th generation (2G, 3G, 4G, or 5G) cellular technology, or the like. Network access technologies can allow wide area coverage for devices, such as user devices with varying degrees of mobility, for example.

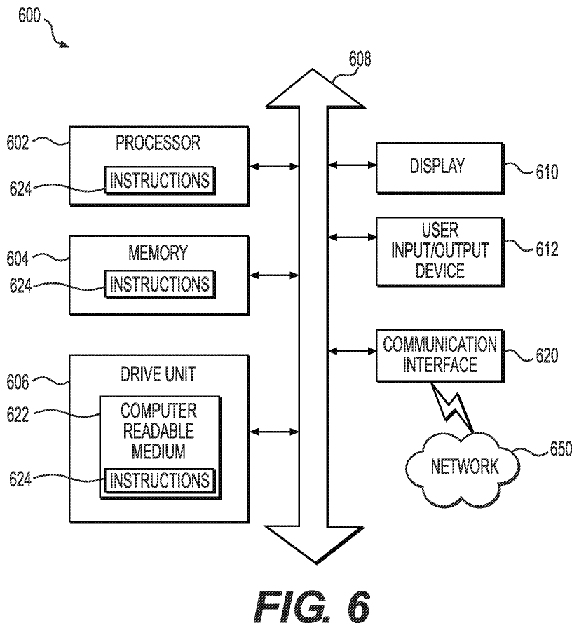

The user device 104 may include any electronic equipment, controlled by a processor (e.g., central processing unit (CPU)), for inputting information or data and displaying a user interface. A computing device or user device can send or receive signals, such as via a wired or wireless network, or can process or store signals, such as in memory as physical memory states. A user device may include, for example: a desktop computer; a mobile computer (e.g., a tablet computer, a laptop computer, or a notebook computer); a smartphone; a wearable computing device (e.g., smart watch); or the like, consistent with the computing device shown in FIG. 6.

The server device 106 may include a service point which provides, e.g., processing, database, and communication facilities. By way of example, and not limitation, the term “server device” can refer to a single, physical processor with associated communications and data storage and database facilities, or it can refer to a networked or clustered complex of processors, such as an elastic computer cluster, and associated network and storage devices, as well as operating software and one or more database systems and application software that support the services provided by the server. The server device 106, for example, can be a cloud-based server, a cloud-computing platform, or a virtual machine. Server devices 106 can vary widely in configuration or capabilities, but generally a server can include one or more central processing units and memory. A server device 106 can also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

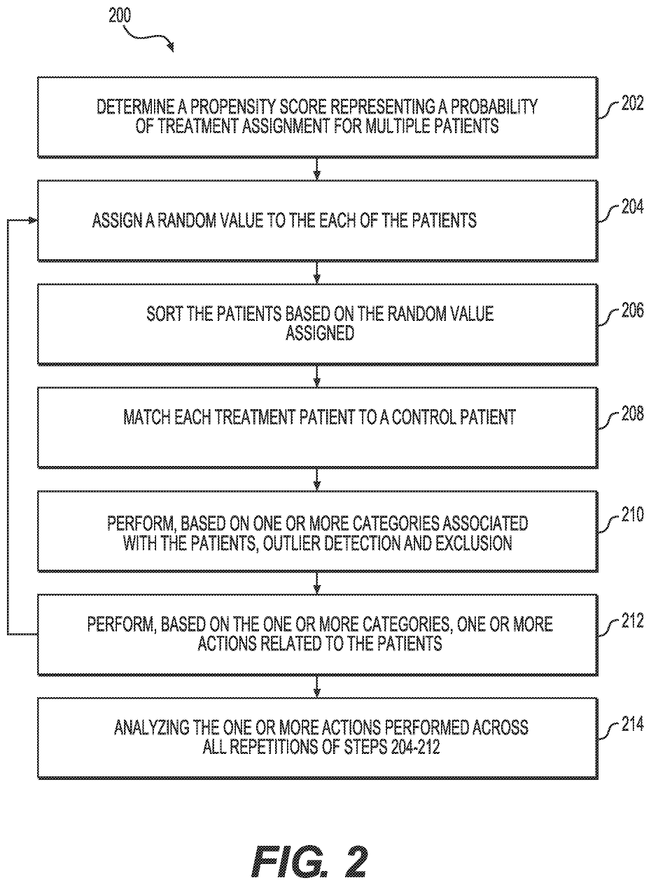

The intervention evaluation platform 108 may include a computing platform hosted on one or more server devices 106. The intervention evaluation platform 108 may provide certain modules, databases, user interfaces, and/or the like for performing certain tasks, such as data processing and/or analysis tasks. For example, the intervention evaluation platform 108 may perform the method 200 illustrated in FIG. 2 below. In some embodiments, a user may use a user device 104 to access one or more user interfaces associated with the intervention evaluation platform 108 to control operations of the intervention evaluation platform 108.

The data store 110 may include one or more non-volatile memory computing devices that may store data in data structure, databases, and/or the like. The data store 110 may include or may be hosted on one or more server devices 106. In some embodiments, the data store 110 may store data related to and/or used for intervention evaluation, output from the intervention evaluation platform 108, and/or the like.

FIG. 2 depicts a flowchart illustrating an exemplary method 200 for evaluating an intervention, according to one or more embodiments. For example, the intervention evaluation platform 108 may perform the method 200. The intervention may include a treatment group of a plurality of treatment patients and a control group of a plurality of control patients.

At step 202, the method 200 may include determining a propensity score for each of multiple patients in an intervention. The propensity score may represent a probability of treatment or treatment assignment, given the observed characteristics or covariates. Determining the propensity score includes generating a propensity score model, which may be a machine learning model including a logic model or any other binary dependent variable classification model. For example, the intervention evaluation platform 108 may determine the propensity score and/or generate the propensity score model. The generated propensity score model may be configured to determine a probability, for each patient, of a treatment assignment based on observed baseline characteristics of the patient. The probability may be expressed as a propensity score. The propensity score model may be used in a healthcare observational study to estimate an effect of receiving treatment when random assignment of treatments to subjects is not feasible.

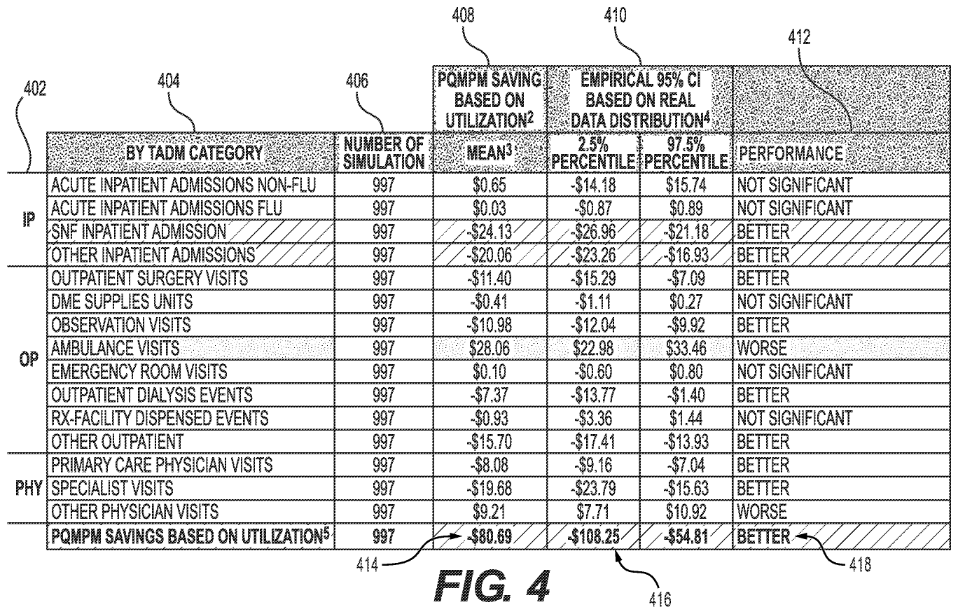

At step 202, the intervention evaluation platform 108 and/or the generated propensity score model may receive patient information, utilization/cost data, and/or covariates relating to each of the patients to determine their propensity scores. For example, the intervention evaluation platform 108 may receive information regarding the patient's age, sex, location, education level, socioeconomic status, income level, height, weight, identity, disease, comorbidities, or other variables or information related to a disease, outcome, utilization and cost, etc. Patient information may also include information associated with a trend analysis data model (TADM) level or category (illustrated in FIG. 4, such as ambulance visits, etc.). Patient information may also include information related to a medical claim, medical codes related to diagnoses and/or procedures associated with a patient (e.g., International Classification of Diseases (ICD) codes, Current Procedural Terminology (CPT) codes, drug codes, etc.). The patient information received at the intervention evaluation platform 108 may be used to generate the propensity score model and/or in running the propensity score model to determine the propensity scores. The received patient information may also be used in performing an activity associated with the patients (e.g., a monetization utilization or a cost savings estimate).

The method 200 may include, at step 204, assigning a random value, number, or variable to each of the multiple patients. For example, the intervention evaluation platform 108 may assign the random value (e.g., 0.003, 0.001, etc.) to each of the patients. Step 204 may include using one seed (e.g., a first seed) to assign the random value. For example, the intervention evaluation platform 108 may determine and/or select the first seed, and use the first seed to assign the random value. As will be described in more detail later, the method 200 (or at least steps 204, 206, 208, 210, and/or 212) may be repeated. When repeating step 204, a different seed (e.g., a second seed) in a different iteration or simulation (e.g., second iteration or simulation) may be used to assign the random value. For example, in repeating step 204 in a second iteration or simulation, the intervention evaluation platform 108 may determine and/or select the second seed, and use the second seed to assign (a likely different) random value for each patient.

In some examples, a random value is assigned to every patient (both treatment patients and control patients) in the intervention. In other examples, step 204 may include assigning a random value to some, but not all, of the multiple patients, or enough of the patients to impact an order in step 206. In these examples, those assigned a random value may be an “assignment group,” which may include at least one of the treatment group or the control group. For example, step 204 may include assigning a random value to each of the treatment patients in the treatment group, but not the control patients in the control group. Alternatively, step 204 may include assigning a random value to each of the control patients in the control group, but not the treatment patients in the treatment group. These techniques may be combined in a same study. For example, a first simulation may use a default order of both treatment patients and control patients, a second simulation may re-sort treatment patients but not control patients, a third simulation may re-sort control patients but not treatment patients, and a fourth simulation may re-sort both treatment patients and control patients.

The method 200 may include, at step 206, sorting or determining an order of the patients based on the random value assigned to each of the patients. For example, the intervention evaluation platform 108 may sort the patients based on the random values assigned in step 204 (e.g., in increasing or decreasing order of the random value assigned). The sorting in step 206 may also be based on the propensity scores determined in step 202. For example, the sorting in step 206 may include determining a final or secondary score for each patient based on the assigned random value and also the propensity score.

The sorting in step 206 may change a data order of patients and/or reorder the patients. Thus, instead of using a default or initial order in step 208 to match treatment and control patients, the techniques disclosed herein may reorder data (e.g., patients) before propensity score matching. Because many patients may have a same or similar probability of being in the treatment group and/or propensity score, the randomly assigned values may differentiate or distinguish these patients. When repeating step 206 in the next simulation, the order of patients may change because the random values and/or seeds on which the order is based may change.

In some examples, step 206 may include sorting the treatment patients based on their assigned random value, and also sorting the control patients based on their assigned random value. Where only some of the patients were assigned a random value (e.g., just the treatment patients or just the control patients), step 206 may include sorting only those patients that were assigned the random value (e.g., sorting just the treatment patients while leaving the control patients in a default order, or sorting just the control patients while leaving the treatment patients in a default order). An advantage of assigning random values to and sorting both treatment patients and control patients, however, is that there are more possibilities for different orders and/or matches.

The method 200 may include, at step 208, matching each treatment patient to at least one control patient. Matching at step 208 may create a set of multiple pairs or matches, where each match includes one treatment patient and at least one control patient. As used herein, the word “match” may include pairs of treatment and control patients and also include larger groups that include one or more treatment patients and one or more control patients. The patients in the treatment group may be matched to patients in the control group who have similar characteristics. For example, the intervention evaluation platform 108 may match each treatment patient to at least one control patient using one or more machine learning models. As another example, the generated propensity score model may also perform matching (in addition to, for example, assigning the random value and sorting the treatment patients).

Matching step 208 of matching patients in the treatment group to those in the control group who have similar characteristics may be based on determined propensity scores. During the matching at step 208, a treatment patient appearing first in the sorted order may be matched to a first control patient (relative to an order of the control patients) having a closest or most similar propensity score. Where the control group includes two or more patients having a same propensity score or most similar propensity score to this first treatment patient, the first treatment patient may be matched with the control patient appearing first in the order of the control patients. The control patient matched to this first treatment patient is removed from the control group of control patients. A next treatment patient ordered later or after the initial or previous treatment patient will be matched to a next remaining control patient having a closest or most similar propensity score. Alternatively or in addition hereto, matching may be performed by starting with the control patients, and matching a first control patient to a first treatment patient (relative to an order of treatment patients) having a closest or most similar propensity score. Where the treatment group includes two or more patients having a same propensity score or most similar propensity score to this first control patient, the first control patient may be matched with the treatment patient appearing first in the order of the treatment patients.

In an example where the each control patient was assigned a random value and the control patients were sorted, while the treatment patients were not assigned a random value and/or left in a default order, assigning step 204 may assign a random value to each of the control patients, the sorting in step 206 may sort the control patients based on their assigned random values, and the matching step 208 may match a control patient to a similar treatment patient based on the sorted order of control patients and propensity scores. In an example where each treatment patient was assigned a random value and the treatment patients were sorted, while the control patients were not assigned a random value and/or left in a default order, assigning step 204 may assign a random value to each of the treatment patients, the sorting in step 206 may sort the treatment patients based on their assigned random values, and the matching step 208 may match a treatment patient to a similar control patient based on the sorted order of treatment patients and propensity scores.

Step 208 may include performing a 1:1 match to create a pair (e.g., using a greedy matching or nearest neighbor algorithm) or a 1:N match (e.g., including one treatment patient matched with N control patients). In a next simulation, at least some of the matches may change in step 208 because the order has changed. As used herein, the word “match” may include 1:1 matches or pairs and 1:N matches. The word “match” is not meant to exclude pairs.

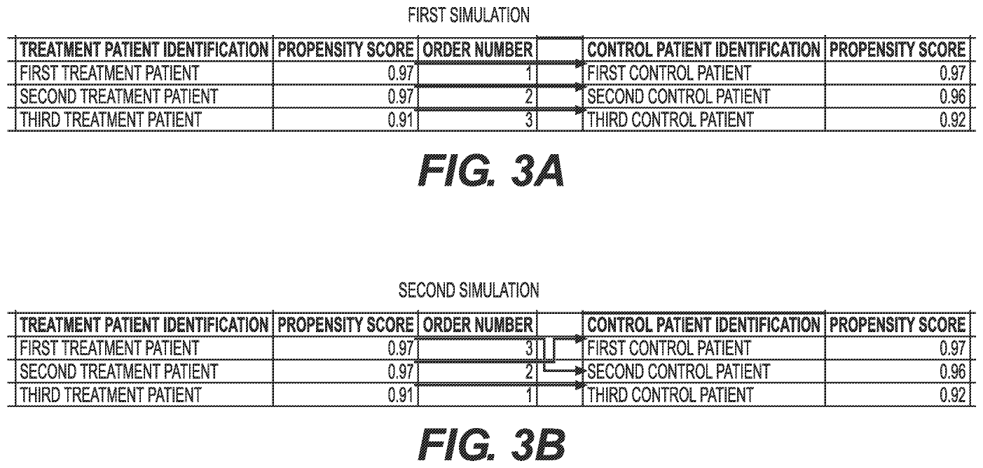

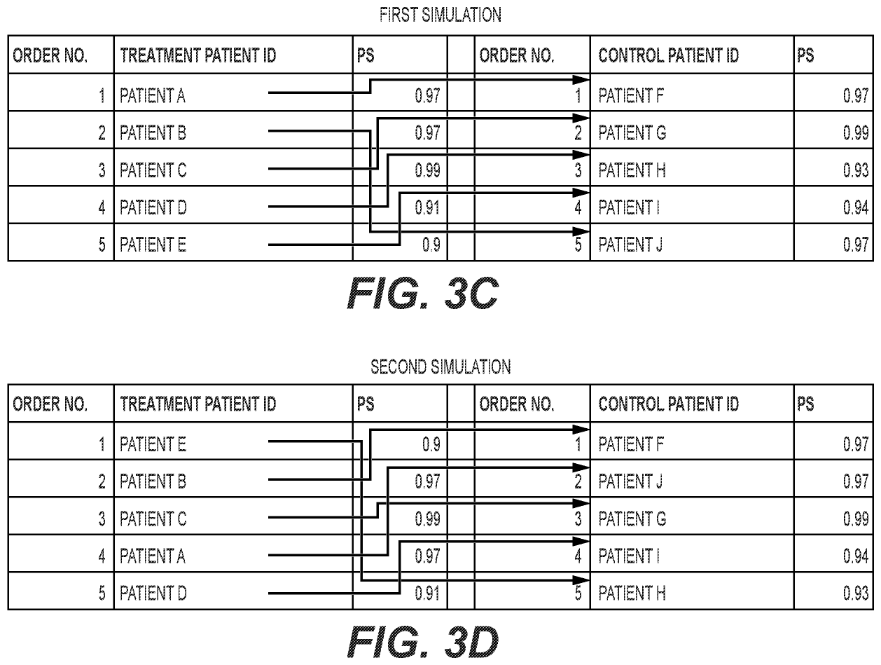

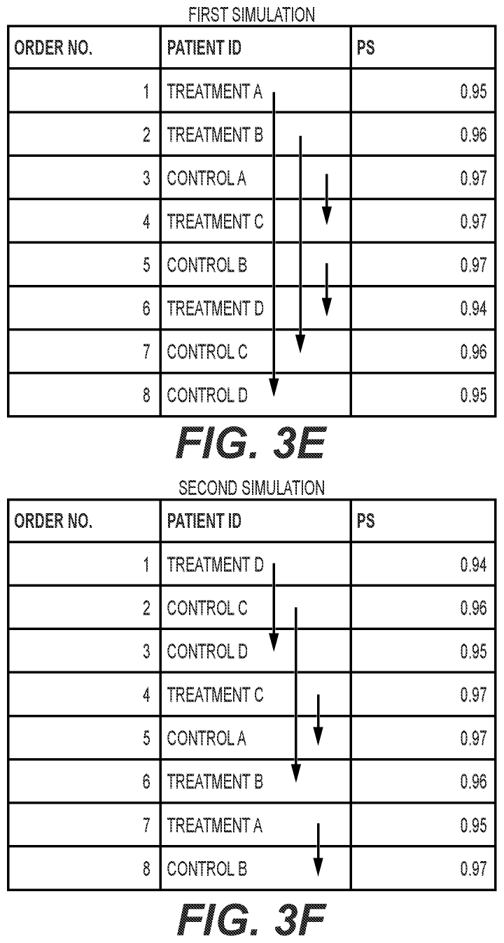

FIGS. 3A and 3B provide a simplified example where treatment patients are assigned a random value and resorted as compared to a default order, while control patients remain in a default order. Referring to FIGS. 3A and 3B, the plurality of treatment patients may include a first treatment patient, a second treatment patient, etc. and the plurality of control patients may include a first control patient, a second control patient, etc. FIGS. 3A and 3B show an example including three treatment patients and three control patients to simplify a matching example in different simulations, but aspects disclosed herein are not limited.

During step 202 of determining the propensity score, the first treatment patient may be determined to have a first propensity score and the second treatment patient may be determined to have a second propensity score. The first control patient may have a third propensity score (which may have been determined in step 202, or alternatively may have been determined prior to or after step 202). The second control patient may have a fourth propensity score (which may have been determined in step 202, or alternatively may have been determined prior to or after step 202).

In some examples, among all propensity scores of the control patients (or at least all remaining propensity scores of the control patients, if some matching has already occurred), both of the first and second propensity scores of the first and second treatment patients may be closest to the third propensity score of the first control patient. This similarity may occur when, for example, the first, second, and third propensity scores all have a same value (as exemplified in FIGS. 3A and 3B, where the first propensity score is 0.97, the second propensity score is 0.97, and the third propensity score of the first control patient is 0.97); when at least the first and second propensity scores are very similar (e.g., the first propensity score is 0.964, the second propensity score may is 0.969, and the third propensity score is 0.95 and is the highest among all propensity scores for the control patients); and/or when the third propensity score is similar to both the first and second propensity scores (e.g., the first propensity score is 0.970, the second propensity score is 0.950, and the third propensity score is 0.960 when the other propensity scores in the control group consist of 0.999, 0.935, and 0.875). In the example of FIGS. 3A and 3B, the first and second treatment patients may have the same propensity score of 0.97 as the first control patient, while the third treatment patient may have a propensity score of 0.91, the second control patient may have a propensity score of 0.96, and the third control patient may have a propensity score of 0.92.

During assigning the random value in step 204, the first treatment patient may be assigned a first value and the second treatment patient may be assigned a second value. The second value may be different from the first value. The third treatment patient may be further assigned a third value, which may be different from the first and second values.

During sorting in step 206, the first treatment patient may be ordered before or after the second treatment patient based on the value assigned in step 204. For example, the first value may be less than the second value, and the treatment patients may be ordered in ascending order, so the first treatment patient may be ordered before the second treatment patient. In other examples, the treatment patients may be ordered in descending order, so the second patient treatment may be ordered before the first treatment patient. In some examples, the value assigned may be combined with the propensity score (e.g., multiplied or added) to yield a final score, and the order may be assigned based on the final score. A determination of any such final score may not be limited. In some examples, the first simulation may be based on a default or initial order and may not include a randomly assigned value until the second simulation.

In FIG. 3A exemplifying the first simulation, the first treatment patient is ordered before the second treatment patient, which is ordered before the third treatment patient (as indicated by the “order number” column in FIG. 3A). This order may be a default order, or may be a first random order based on a random value assignment in step 204. In FIG. 3B exemplifying the second simulation, the third treatment patient is ordered before the second treatment patient, which is ordered before the first treatment patient.

During matching in step 208, if the first treatment patient is ordered before the second treatment patient (as in the first simulation in FIG. 3A), even if both the first and second propensity scores are closest to the third propensity score among all other (remaining) propensity scores (here, the first and second propensity scores of 0.97 are closer to the third propensity score of 0.97 than the fourth propensity score of 0.96), the first treatment patient, and not the second treatment patient, may be matched with the first control patient having the third propensity score due to the first treatment patient being ordered before the second treatment patient. The second treatment patient may be matched with the second control patient having the fourth propensity score. Here, the second propensity score may be closest to, among all remaining propensity scores among the control patients after the third propensity score is removed, the fourth propensity score. The third treatment patient may be matched with the remaining control patient having the best propensity score, which may be the third control patient.

In this example where the first and second propensity scores are closest to the third propensity score among all other propensity scores in the control group, had the second treatment patient been assigned a random value in step 204 causing the second treatment patient to be ordered before the first treatment patient in sorting step 206 (as in the second simulation, shown in FIG. 3B), then in step 208, the second treatment patient, and not the first treatment patient, would have been matched with the first control patient having the third propensity score.

FIGS. 3A and 3B illustrate how an order may change the pairs. When the method 200 (or at least steps 204, 206, and 208) is repeated (or in some examples, first performed) in a next iteration or simulation, because the order of the first and second treatment patients is based on random assignments, the second treatment patient may be ordered before the first treatment patient, which may yield a different match. After step 208 of matching is performed to yield the matches in FIG. 3A, the method 200 may include repeating step 204 of assigning the random value. During repeating step 204 of assigning the random value, the first treatment patient may be assigned a fourth value and the second treatment patient may be assigned a fifth value. The fourth value may be different from the fifth value. The third treatment patient may be assigned a sixth value, which may be different from the fourth and fifth values.

The method 200 may include repeating step 206 of sorting based on the repeated assigning of random values (e.g., the fourth, fifth, and sixth values). As exemplified in FIG. 3B, during repeating the sorting in step 206, the second treatment patient may be ordered before the first treatment patient. The third treatment patient, may be ordered before the first and second patients, but a similar matching result in step 208 as shown in FIG. 3B would occur if the third treatment patient had been ordered after the first and second patients (based on the exemplified propensity scores).

The method 200 may include repeating the matching step 208 based on the new order. As shown in FIG. 3B, during repeating the matching, the second treatment patient may be matched with the first control patient.

In this second simulation of FIG. 3B, the third treatment patient may be still matched with the third control patient, as the third control patient has a closest propensity score to that of the third treatment patient. However, because the second treatment patient is ordered before the first treatment patient, the second treatment patient is matched with the first control patient, while the first treatment patient is matched with the control patient having the best remaining propensity score, which is the second control patient having the fourth propensity score (0.96 in FIGS. 3A and 3B). An order in which the matching occurs in step 208 may therefore change the resulting matches.

Thus, across multiple iterations of method 200, where the order of the patients changes in step 206, different and/or new pairs may be created, which may yield (over many iterations) more data and/or comparisons that can be averaged for more accurate results. In some examples, the random value assignment in step 204 and sorting step 206 may be omitted only in one (e.g., the first) simulation, while steps 204 and 206 may be included in subsequent iterations. In some examples, the assignment of random values in step 204, the sorting in step 206, and the matching in 208 may occur within categories (e.g., trend analysis data model or TADM categories) associated with the treatment patients and/or pairs.

FIGS. 3C and 3D show an example where both treatment patients and control patients are re-sorted. Referring to FIG. 3C, in a first simulation, when matching based on the orders of the treatment and control groups, Patient A may be matched with Patient F, Patient B may be matched with Patient J, Patient D may be matched with Patient H, and Patient E may be matched with Patient I. Referring to FIG. 3D, in a second simulation, an order of both the treatment patients and the control patients may be changed. As seen in FIG. 3D, this new order may result in Patient A being matched with Patient J, Patient B being matched with Patient F, Patient D being matched with Patient I, and Patient E being matched with Patient H. Thus, an order may change a makeup of pairs, providing more pairs having same or similar propensity scores to analyze.

Although FIGS. 3A-3D visually depict a treatment group and control group on different sides, when assigning random values and sorting both treatment groups and control groups, patients in the treatment group and control group may be mixed (i.e., sorted and ordered) together. For example, in FIG. 3E, the treatment and control patients may be ordered together, and matching may occur in order to match one type of patient (e.g., treatment or control) with a first of the other type of patient (e.g., control or treatment) having a same or most similar propensity score. In FIG. 3F, the patients may be reordered based on a randomly assigned value, and the resulting pairings may be different.

Referring back to FIG. 2, the method 200 may include, at step 210, removing one or more outlier pairs or matches among a set of matched pairs or matches created in step 208. Outlier pairs or matches may be identified and/or determined by performing outlier detection and exclusion for the multiple patients. Step 210 and/or the performing outlier detection and exclusion may be performed before, after, during, or concurrently with step 212 described later, and may be based on the matching in step 208, a comparison of a treatment patient to the control patient in each match, a value determined based on the match and/or a value associated with the match, one or more categories associated with the multiple patients, characteristics associated the patients, received patient data, a probability that a patient will be assigned to the treatment group, etc. Removing the one or more outlier pairs or matches from the set of matched pairs or matches created in step 208 may create a subgroup of pairs or matches.

For example, each treatment patient may be associated with a certain or initial value, such as a cost for treatment of the treatment patient, a cost associated with an event of the treatment patient, a cost based on a total per member per month (PMPM) cost, or a cost associated with a category of the treatment patient (e.g., a TADM category). This value may be received and/or determined at step 202 of determining propensity scores, but may not necessarily be factored into the determination of the propensity score. For example, the PMPM cost may be provided as part of the propensity score model and/or received by the propensity score model. Alternatively, this value may be received and/or determined at step 210. Some of the treatment patients may be outliers based on this associated initial value. After matching in step 208, an outlier pair may be any pair that includes an outlier patient (e.g., an outlier treatment patient or an outlier control patient), and such outlier pairs may be removed. Removal of the outlier pairs may occur after matching in step 208, as removing before may create an unequal number of treatment patients and control patients.

Outlier patients may be determined and/or detected by determining one or more patients having an associated value within a highest and lowest percentage among all values associated with the patients (e.g., patients associated with a value in the top and/or bottom 0.5% of all associated values) and excluding the pairs among the multiple created matched pairs in step 208 that include any of the detected highest and lowest value patients. Alternatively, outlier treatment patients may be determined by determining patients having an initial value that is a certain number of standard deviations from a mean initial value. Aspects disclosed herein are not limited to how an outlier is determined or statistical processes used to determine outliers (e.g., using interquartile range, etc.). An outlier pair may be any pair including an outlier patient.

As an alternative to using an initial value associated with the patient (e.g., cost), outliers may be based on other characteristics or associated with the patients. For example, a patient having an outlier height, age, or weight may be excluded in some examples.

As an alternative to determining outlier treatment patients, in some examples, outlier pairs or matches may be determined based on a comparison of the treatment patient to the control patient. In this example, step 210 may be performed concurrently or between certain actions of analyzing the matched pairs in step 212. For example, after determining, in step 212, a difference between a value associated with the treatment patient and a value associated with the control patient, this difference may be compared to the differences determined in other pairs or matches, and outlier pairs or matches (e.g., top or bottom 0.5%) may be excluded from further analysis in step 212.

In a cost savings estimate context, the intervention evaluation platform 108 may determine or identify the treatment patients having a cost based on a total per member per month (PMPM) cost that falls within the top 0.5% of costs based on a total per member per month (PMPM) cost, and remove or exclude, in step 210, any matched pairs or groups that include these identified patients such that analysis in step 212 excludes these removed pairs or matches. The cost based on a total PMPM cost may be received and/or determined for each treatment patient at step 202 and/or step 210. In some examples, outliers may not be detected in step 210, and no pairs or matches may be removed.

The method may include, at step 212, performing one or more actions related to the patients and/or matched pairs. Step 212 may be based on the subgroup of pairs or matches created in step 210 after removing any outlier pairs or matches. The intervention evaluation platform 108 may perform one or more actions related to the patients and/or the subgroup of pairs or matches. For example, the one or more actions may include comparing, within each match, a value (e.g., cost) associated with the treatment patient and a value (e.g., cost) associated with the control patient, and determining a difference between the value associated with the treatment patient and the value associated with the control patient. When the values are costs (e.g., cost based on a total PMPM cost), this difference may reflect and/or be used to calculate a cost savings estimate. The one or more actions may further include determining an average difference among all matched groups or pairs. The one or more actions may include other statistical calculations based on values associated with the treatment patients and/or other characteristics associated with the treatment patients, and aspects disclosed herein are not limited to the described statistical analyses of each match or of the group of matches in a same simulation or iteration. Performing the one or more actions may be based on one or more categories.

For example, step 212 may include determining or calculating a savings or a savings estimate. Determining the savings may be performed by using monetization of utilization by a trend analysis data model (TADM) category. A total savings may be a sum of savings of each TADM category associated with the treatment patients and/or matched groups or pairs. Monetization of utilization may be performed at each TADM category and/or category level.

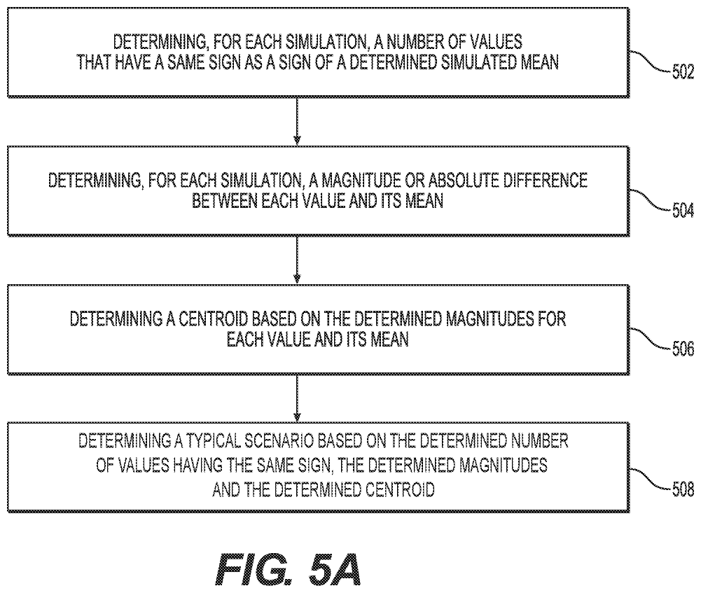

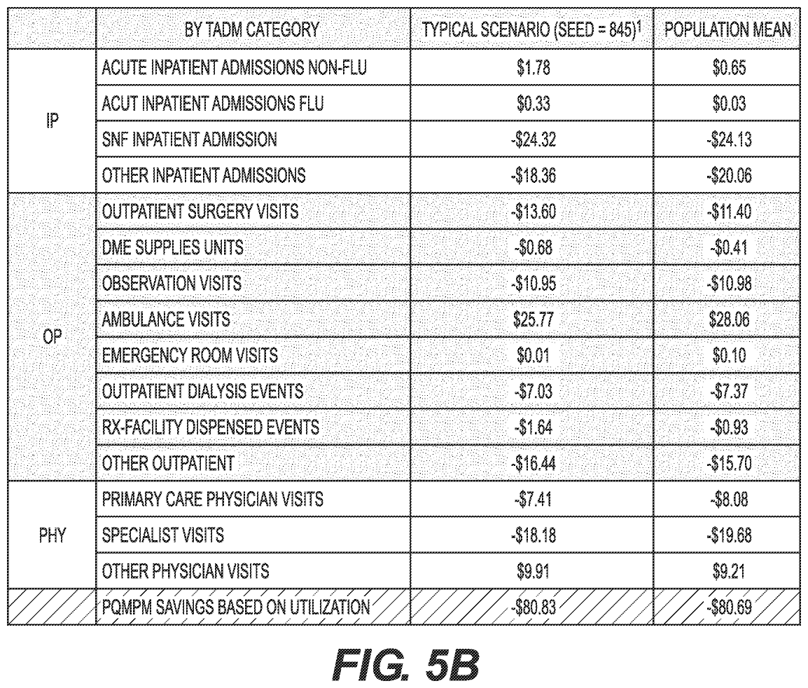

For example, the intervention evaluation platform 108 may determine or calculate a delta, difference, or change in an observational period between the treatment group and the control group on a utilization. The intervention evaluation platform 108 may determine a treatment group unit cost in an observational study period. The actions and/or determinations (e.g., the total savings and/or savings at each TADM category) may be saved and/or stored (e.g., to an electronic storage). In addition, the actions and/or determinations (e.g., the total savings and/or savings at each TADM category, etc.) may be output (e.g., on a user interface such as an electronic display or to electronic or digital storage or memory). For example, all simulated data may be saved to an electronic storage system. The stored simulated data may be used to calculate the mean and empirical 95% CI values shown in FIG. 4 and/or to identify typical scenarios and/or primary or secondary outcomes, as shown in FIG. 5. Tables similar to those shown in FIGS. 4 and 5B may be output onto a display and/or manipulated by a user. Other data and/or analyses based on the simulated data may also be displayed. For example, one or more plots based on matches or pairs and propensity scores may be output.

As previously described, the method 200 may include repeating one or more of steps 204 through 212. Steps 204-212 may be repeated a predetermined number N times or iterations (e.g., 1000 times, in a range of 800-1100 times, etc.), and each time step 204 is repeated, a different seed (N) may be used. The Nth time step 204 is repeated, the intervention evaluation platform 108 may select an Nth seed, and use that Nth seed to assign (a likely different) random value for each patient. Thus, aspects disclosed herein may determine N (e.g., 1000) cost saving estimates and/or comparisons by TADM category, plus total savings. All N repetitions use the same generated propensity score model (such as the generated propensity score model in step 202) and use the same multiple treatment and/or control patients in the intervention. Aspects disclosed herein may therefore repeat propensity score matching multiple times on a same original study cohort.

The method 200 may further include a step 214 of analyzing the one or more actions performed across all repetitions of steps 204 through 212. Step 214 may include statistically analyzing the results of all N runs. In either step 212 and/or 214, the intervention evaluation platform 108 may statistically analyze the result of each run, and step 214 may include analyzing all runs. Step 212 and/or Step 214 may include, for each N run, comparing a bell or distribution curve of the treatment patients to a bell or distribution curve of their matched control patients.

Step 214 may include determining a set of values, a distribution of values, means, composite or summary values, etc. Step 214 may include determining a set of and/or distribution of values for the treatment patients and/or matches based on values associated with the treatment patients, the control patients, and/or within each matched pair or match (e.g., a comparison of a value, such as cost, of the treatment patient with the value of its matched control patient).

For example, step 214 may include determining a mean savings by TADM category, plus total savings, based on the results of the N repeated runs. Savings may be based on a difference between a cost or value of the treatment patient and a cost or value of the control patient, or based on utilization monetization. Step 214 may also include determining a predetermined confidence level (CI). For example, the intervention evaluation platform 108 may determine and/or calculate an empirical 95% CI. Exemplary determinations and outputs of this statistical analysis are explained in more detail with reference to FIG. 4.

The statistical analysis and/or determinations (e.g., the total savings, mean savings at each TADM category, and/or empirical 95% CI) may be saved and/or stored (e.g., to an electronic storage). In addition, the statistical analysis and/or determinations (e.g., the total savings, mean savings at each TADM category, and/or empirical 95% CI) may be output (e.g., to an electronic display). For example, a summary of the statistical analysis may be output as a table, like the table in FIG. 4, and step 214 may include all of the calculations and/or determinations described in connection with FIG. 4. These results may be used to determine a most typical case scenario, as described in more detail in FIG. 5A, so that a user may accurately evaluate the intervention and/or concisely present results when marketing or presenting the intervention.

FIG. 4 illustrates an example simulation of utilization monetization using the method 200 to evaluate a performance of an intervention. FIG. 4 may be an exemplary output of a summary of results and/or analysis after running the propensity score model N times. Referring to FIGS. 2 and 4, in the example of FIG. 4, a number N of simulations or iterations 406 was 997 simulations, indicating that steps 204-212 were repeated 997 times. At least the matching step 208 and/or the outlier detection 210 may be performed within different categories or levels (e.g., a TADM category). Each of the 997 iterations (or simulations) represent one matched cohort using a different seed during the assignment step 204, where outliers (e.g., TADM outliers) are excluded after each match.

For example, after matching pairs or matches in step 208, outlier matches or pairs having higher and/or lower costs may be further analyzed in step 210. If the match (or one of the patients in the match or pair) is different enough, the match may be excluded from the entire group of matches or pairs. In excluding a match, a reason why the cost is different may be assessed. A match may be excluded if the treatment patient and/or the control patient in the match is statistically very high (e.g., in one TADM category). In excluding outliers, the remaining set of matches (or subgroup) on which to run the propensity score matching may represent more normal or typical costs (e.g., in associated TADM categories, or at least in certain TADM categories of interest).

A TADM category may be a grouping logic used to address and/or roll up medical claims. There may be one or more TADM levels or large categories 402 and a plurality of TADM categories 404 at each level, but aspects disclosed herein are not limited. Step 212 may detect outliers in each TADM level and/or each TADM category. As an alternative to multiple TADM levels 402 and multiple TADM categories 404 at each level, the TADM levels 402 may be omitted (e.g., if all TADM categories 404 belong to a same level).

As an example, the TADM levels 402 may include inpatient (IP), outpatient (OP), or physician (PHY). The plurality of TADM categories 404 in the IP level may include acute inpatient admissions non-flu, acute inpatient admissions flu, skilled nursing facility (SNF) inpatient admission, or other inpatient admissions in the IP level. The plurality of TADM categories 404 in the OP level may include outpatient surgery visits, durable medical equipment (DME) supplies units, observation visits, ambulance visits, emergency room visits, outpatient dialysis events, prescription or RX-facility dispensed events or medication-facility dispensed events, and other outpatient visits or events. The plurality of TADM categories 404 in the PHY level may include primary care physician visits, specialist visits, or other physician visits or checkups.

Each iteration of running the propensity score model may yield a per qualified member per month (PQMPM) saving based on utilization for each TADM category 404. Here, “qualified member” may mean qualified for the intervention or study. The propensity score model and/or intervention evaluation platform 108 may determine, for each TADM category 404, a mean value for the PQMPM saving based on utilization 408 based on all 997 simulations. Here, a negative value may indicate that money was saved or that a cost was less relative to a control value (e.g., based on the costs and/or values associated with control patients and/or the control group), while a positive value may indicate that money was not saved or a more expensive value relative to the control value. For example, in the example of FIG. 4, the propensity score model calculated that, for SNF inpatient admissions, the mean PQMPM saving based on utilization 408 was −$24.13, reflecting a savings or lower cost by $24.13. This saving mean may represent a savings or difference of a treatment group as compared to the control group over a duration of an observation year (Year 2). In some examples, this saving mean may be compared to values from another (e.g., a previous or control) year, and may reflect and/or be at least partially calculated based on a difference (as opposed to a total cost).

For example, the values for the mean PQMPM saving based on utilization 408 (e.g., −$24.13 for SNF inpatient admissions) may be determined or calculated in the following steps. In step 1, a Year 2 Treatment Group utilization per thousand qualified member per month (PTQMPY) may be determined, a Year 2 Control Group utilization (PTQMPY) may be determined, and a utilization difference in Year 2 may equal the Year 2 Treatment Group utilization (PTQMPY) minus the Year 2 Control Group utilization (PTQMPY). In step 2, the Treatment Group Year 2 PQMPM cost may be determined, and the Treatment Group Year 2 UNIT cost (PQMPM) may be calculated by dividing the Treatment Year 2 PQMPM cost by the Treatment Year 2 PTQMPY utilization times 12000 (i.e., Treatment Year 2 UNIT cost (PQMPM)=[Treatment Year 2 PQMPM cost/Treatment Year 2 PTQMPY utilization]*12000. Since PTQMPY represents a per thousand qualified member per month value, multiplying by 12000 brings the value to a per qualified member per month (PQMPM) value. In step 3, the Treatment Group Year 2 cost saving (PQMPM) may be calculated by multiplying the utilization difference in Year 2 (PTQMPY) from step 1 by the Treatment Group Year 2 UNIT cost (PQMPM) from step 2 and dividing by 12000 (i.e., Treatment Group Year 2 cost saving (PQMPM)=[utilization difference in Year 2 (PTQMPY) (from step 1)*Treatment Group Year 2 UNIT cost (PQMPM) (from step 2)]/12000.

The propensity score model and/or intervention evaluation platform 108 may determine, for each TADM category 404, an empirical 95% saving based on utilization 410 based on real data distribution of all 997 simulations. The empirical 95% CI may include a value for the 2.5% percentile and a value for the 97.5% percentile.

The propensity score model and/or intervention evaluation platform 108 may determine or evaluate, for each TADM category 404, an overall performance 412 of the intervention. This performance 412 may be given a value or assigned a category (e.g., better, worse, not significant), which may be based on the mean value for the PQMPM saving based on utilization 408 and/or the empirical 95% saving based on utilization 410. For example, if, for a given TADM category 404, the mean value for the PQMPM saving based on utilization 408 is a negative number (e.g., −$24.13 for SNF Inpatient Admission) and the empirical 95% saving based on utilization 410 also includes all negative values (i.e., both the value for the 2.5% percentile and the value for the 97.5% percentile are negative), the performance 412 for that given TADM category 404 may be determined to be “better” as compared to a control value. Alternatively or in addition thereto, the propensity score model and/or intervention evaluation platform 108 may determine or evaluate that the mean value for the PQMPM saving based on utilization 408 is “better” based on being higher than a single threshold or first threshold (e.g., “higher” threshold).

If, for a given TADM category 404, the mean value for the PQMPM saving based on utilization 408 is a positive number (e.g., $28.06 for Ambulance Visits) and the empirical 95% saving based on utilization 410 also includes all positive values (i.e., both the value for the 2.5% percentile and the value for the 97.5% percentile are positive), the performance 412 for that given TADM category 404 may be determined to be “worse.” Alternatively or in addition thereto, the propensity score model and/or intervention evaluation platform 108 may determine or evaluate that the mean value for the PQMPM saving based on utilization 408 is worse based on being lower than the single threshold or a second threshold (e.g., “lower” threshold).

If, for a given TADM category 404, the empirical 95% saving based on utilization 410 includes both negative and positive values (e.g., the value for the 2.5% percentile is positive and the value for the 97.5% percentile is negative, or vice versa) the performance 412 for that given TADM category 404 may be determined to be “not significant.” Alternatively or in addition thereto, the propensity score model and/or the intervention evaluation platform 108 may consider a magnitude of the mean value for the PQMPM saving based on utilization 408 (e.g., that the mean value for the PQMPM saving based on utilization 408 is a relatively lower positive or negative number such as $0.03 for Acute Inpatient Admissions Flu, or −$0.41 for DME Supplies Units, where relatively lower may be based on one or more thresholds). to evaluate performance. In an output table to an electronic display, rows with a performance 412 determined to be “better” may be highlighted in a first color (e.g., green), while rows with a performance 412 determined to be “worse” may be highlighted in another color (e.g., red).

The propensity score model and/or intervention evaluation platform 108 may determine or evaluate, for each simulation, a mean value for the PQMPM saving based on utilization 414 and mean values for empirical 95% CI 416 based on the real data distribution across all TADM categories 404 (including values for 2.5% percentile and 97.5% percentile). In FIG. 4, these values are shown in the bottom row. The mean value for the PQMPM saving based on utilization 414 may be calculated in each simulation by summing all values for the PQMPM saving based on utilization 408 for each TADM category and/or by summing each TADM category saving, and then calculating this value's mean and empirical 95% CI. The empirical 95% CI (including values for 2.5% percentile and 97.5% percentile) may also be determined based on the real data distribution across all TADM categories 404.

The propensity score model and/or intervention evaluation platform 108 may determine or evaluate an overall performance 418 for the intervention and for all TADM categories 404 combined. This overall performance 418 may be based on the PQMPM savings based on utilization across all TADM categories 404 and/or the empirical 95% CI based on the real data distribution across all TADM categories 404. In the example of FIG. 4, the overall performance 418 was determined to be “better” based on negative values for the mean value for the PQMPM saving based on utilization 414 and for the empirical 95% CI 416.

Thus, aspects disclosed herein may output a table showing a saving mean, its confidence range by TADM category, and a total saving. An analyst, or the computing device (e.g., intervention evaluation platform 108), may decide whether to present the results based on the confidence ranges or the empirical 95% CI. An analyst, or the computing device (e.g., intervention evaluation platform 108), may determine and present a single iteration (e.g., Nth iteration) that reflects the most typical scenario to a client.

FIG. 5A illustrates a method 500 for selecting a typical iteration or scenario (e.g., a particular N value across all simulations), and FIG. 5B illustrates an exemplary typical scenario. Referring to FIGS. 2, 4, 5A, and 5B, the method 500 may include, in step 502, determining, for each simulation, a number of values or criteria (e.g., distribution of values determined in step 214) that have a same sign as a sign of a determined mean (e.g., simulated mean of the distribution of values). A typical simulation will have a large number of values (e.g., as many values as possible) that are the same sign as the mean. Referring back to the table and calculations described with reference to FIG. 4, a typical simulation may have as many PQMPM savings values as possible that are a same sign (e.g., positive or negative) as a mean saving. This mean saving may be for each TADM category, a total savings value, or the final mean value for the PQMPM saving based on utilization determined for all TADM categories 404 combined.

The method 500 may include, at step 504, determining, for each simulation, a magnitude or absolute difference between each value (e.g., distribution value or savings value) and its mean (e.g., simulated mean). A typical simulation will have a small magnitude or absolute difference. Step 504 may include determining a sum of a standardized absolute value of a difference between a savings and its mean.

The method 500 may include, at step 506, determining a centroid based on the determined magnitudes for each value and its mean for all simulations. Step 506 may include determining a proximity or difference value for each category (e.g., TADM value) and total value as compared to the centroid.

The method 500 may include, at step 508, determining one or more typical scenarios based on the determinations in steps 502 through 506. In some embodiments, the typical scenario may be determined based on one or more of the determined values or criteria in steps 502 through 506. A plurality of typical scenarios may be determined and presented on a user interface, and one of the plurality of typical scenarios may be selected for further analysis and/or to present results.

For example, step 508 may include optimizing and/or rerunning steps 502 through 506 such that the determined typical scenario has, in step 502, as many values (e.g., savings) as possible that have a same sign as the mean. In addition, step 508 may include optimizing steps 502 through 506 such that the determined typical scenario has, in step 504, the smallest determined magnitude as possible and/or that a sum of a standardized absolute difference between a savings and its mean is as small as possible. Step 508 may include analyzing all outputs of steps 502 through 506 and/or ranking the determined values or criteria at each step, as the outputs may not have been filtered via thresholds (e.g., requiring a difference of a first threshold or less in determining a sum of a standardized absolute difference between a savings and its mean), allowing for a dynamic analysis without cutoffs or thresholds. Step 508 may include optimizing steps 502 through 506 (e.g., based on rankings for the outputs, determined values, and/or criteria at each step 502-056) such that the determined typical scenario is, in step 506, overall as close (i.e., less of a difference) as possible to the centroid across all categories (e.g., TADM categories) in view of the total value (e.g., total savings) and in multiple dimensions (e.g., 16 dimensions based on 15 TADM categories and total savings) such that there is no cutoff and the determined typical scenario is dynamic. The typical scenario may be representative of all N simulations.

As an example, FIG. 5B may illustrate the typical scenario based on the table shown in FIG. 4. Here, the typical scenario was determined to be simulation number 845, and the scenario savings of −$80.83 are very similar to the population mean and/or total mean of $80.69 (see also FIG. 4).

In some examples, the most representative scenario may be determined for each TADM level, and these scenarios may be combined in one representative model. For example, N=845 may reflect the most typical scenario for a first TADM category, but N=640 may reflect the most typical scenario for a second TADM category. A composite scenario may be provided generated and/or displayed that includes data from N=845 for the first TADM category and data from N=640 for the second TADM category.

Aspects disclosed herein may perform the simulation at the propensity score matching (PSM) model level, or at other levels. Aspects disclosed herein may be used in various aspects of interventions or other program evaluations, and may be adopted in various study designed. In a non-randomized clinic trial (non-RCT) clinic evaluation program, the simulation may not be performed at the PSM model level, and both the treatment group and the control group may be fixed based on an operation. In RCT clinic evaluation program, the simulation may be performed at the PSM model level because the control group may not be fixed and may be sampled from a large cohort. This simulation can be used in matching with or without replacement even if the basic framework is developed based on matching without replacement. Aspects disclosed herein may not be limited to an intervention, program, or clinical context and may be used where multiple iterations or orders of matching are desired for a data set. For example, instead of treatment and control patients, aspects disclosed herein may be used to match a first group of items (e.g., patients) associated with a score (e.g., PSM score) and a second group of items (e.g., patients) associated with a score (e.g., PSM score).

FIG. 6 illustrates an implementation of a computer system that may execute techniques presented herein. The computer system 600 can include a set of instructions that can be executed to cause the computer system 600 to perform any one or more of the methods or computer based functions disclosed herein. The computer system 600 may operate as a standalone device or may be connected, e.g., using a network, to other computer systems or peripheral devices.

In a networked deployment, the computer system 600 may operate in the capacity of a server or as a client user computer in a server-client user network environment, or as a peer computer system in a peer-to-peer (or distributed) network environment. The computer system 600 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 600 can be implemented using electronic devices that provide voice, video, or data communication. Further, while a single computer system 600 is illustrated, the term “system” shall also be taken to include any collection of systems or sub-systems that individually or jointly execute a set, or multiple sets, of instructions to perform one or more computer functions.

As illustrated in FIG. 6, the computer system 600 may include a processor 602, e.g., a central processing unit (CPU), a graphics processing unit (GPU), or both. The processor 602 may be a component in a variety of systems. For example, the processor 602 may be part of a standard personal computer or a workstation. The processor 602 may be one or more general processors, digital signal processors, application specific integrated circuits, field programmable gate arrays, servers, networks, digital circuits, analog circuits, combinations thereof, or other now known or later developed devices for analyzing and processing data. The processor 602 may implement a software program, such as code generated manually (i.e., programmed).

The computer system 600 may include a memory 604 that can communicate via a bus 608. The memory 604 may be a main memory, a static memory, or a dynamic memory. The memory 604 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 604 includes a cache or random-access memory for the processor 602. In alternative implementations, the memory 604 is separate from the processor 602, such as a cache memory of a processor, the system memory, or other memory. The memory 604 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 604 is operable to store instructions executable by the processor 602. The functions, acts or tasks illustrated in the figures or described herein may be performed by the programmed processor 602 executing the instructions stored in the memory 604. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the computer system 600 may further include a display unit 610, such as a liquid crystal display (LCD), an organic light emitting diode (OLED), a flat panel display, a solid-state display, a cathode ray tube (CRT), a projector, a printer or other now known or later developed display device for outputting determined information. The display 610 may act as an interface for the user to see the functioning of the processor 602, or specifically as an interface with the software stored in the memory 604 or in the drive unit 606.

Additionally or alternatively, the computer system 600 may include an input device 612 configured to allow a user to interact with any of the components of system 600. The input device 612 may be a number pad, a keyboard, or a cursor control device, such as a mouse, or a joystick, touch screen display, remote control, or any other device operative to interact with the computer system 600.

The computer system 600 may also or alternatively include a disk or optical drive unit 606. The disk drive unit 606 may include a computer-readable medium 622 in which one or more sets of instructions 624, e.g. software, can be embedded. Further, the instructions 624 may embody one or more of the methods or logic as described herein. The instructions 624 may reside completely or partially within the memory 604 and/or within the processor 602 during execution by the computer system 600. The memory 604 and the processor 602 also may include computer-readable media as discussed above.

In some systems, a computer-readable medium 622 includes instructions 624 or receives and executes instructions 624 responsive to a propagated signal so that a device connected to a network 650 can communicate voice, video, audio, images, or any other data over the network 650. Further, the instructions 624 may be transmitted or received over the network 650 via a communication port or interface 620, and/or using a bus 608. The communication port or interface 620 may be a part of the processor 602 or may be a separate component. The communication port 620 may be created in software or may be a physical connection in hardware. The communication port 620 may be configured to connect with a network 650, external media, the display 610, or any other components in system 600, or combinations thereof. The connection with the network 650 may be a physical connection, such as a wired Ethernet connection or may be established wirelessly as discussed below. Likewise, the additional connections with other components of the system 600 may be physical connections or may be established wirelessly. The network 650 may alternatively be directly connected to the bus 608.

While the computer-readable medium 622 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 622 may be non-transitory, and may be tangible.

The computer-readable medium 622 can include a solid-state memory such as a memory card or other package that houses one or more non-volatile read-only memories. The computer-readable medium 622 can be a random-access memory or other volatile re-writable memory. Additionally or alternatively, the computer-readable medium 622 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 600 may be connected to one or more networks 650. The network 650 may define one or more networks including wired or wireless networks. The wireless network may be a cellular telephone network, an 802.11, 802.16, 802.20, or WiMax network. Further, such networks may include a public network, such as the Internet, a private network, such as an intranet, or combinations thereof, and may utilize a variety of networking protocols now available or later developed including, but not limited to TCP/IP based networking protocols. The network 1050 may include wide area networks (WAN), such as the Internet, local area networks (LAN), campus area networks, metropolitan area networks, a direct connection such as through a Universal Serial Bus (USB) port, or any other networks that may allow for data communication. The network 650 may be configured to couple one computing device to another computing device to enable communication of data between the devices. The network 650 may generally be enabled to employ any form of machine-readable media for communicating information from one device to another. The network 650 may include communication methods by which information may travel between computing devices. The network 650 may be divided into sub-networks. The sub-networks may allow access to all of the other components connected thereto or the sub-networks may restrict access between the components. The network 650 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

Aspects disclosed herein may analyze treatment groups of patients and control groups of patients. In some examples, one or both of these groups may be provided by third parties, outside vendors, internal vendors, outside programs, etc. For example, Kidney Resource Services (KRS), a clinic program to take care of kidney patients may provide control patients and/or the control group, while Somatus, a third party vendor to provide kidney disease patient care, may provide treatment patients and/or the treatment group.

We claim:

1. A computer-implemented method for evaluating programs, the method comprising:
determining a propensity score, using a propensity score model, for each patient among multiple patients, wherein the multiple patients include a plurality of treatment patients and a plurality of control patients, and the propensity score represents a probability of assignment to a treatment group;
assigning a random value to each patient in an assignment group, the assignment group including at least one of the plurality of treatment patients or the plurality of control patients;
sorting the plurality of patients based on the assigned random values;
matching, based on the sorted patients and the determined propensity scores, each treatment patient to a control patient to create multiple matches, each match including one treatment patient and at least one control patient; and
performing, based on the multiple matches, one or more actions related to the multiple patients.

2. The method of claim 1, further comprising repeating assigning the random value, sorting the plurality of patients, and matching, wherein, upon repeating assigning the random value, each patient in the assignment group is assigned a random value that is different from the random value previously assigned.

3. The method of claim 1, wherein assigning the random value is based on a first seed, and wherein the method includes repeating assigning the random value based on a second seed.

4. The method of claim 1, wherein sorting is further based on the determined propensity scores.

5. The method of claim 1, wherein matching is performed in order of the sorted patients.

6. The method of claim 1, further comprising removing one or more outlier matches from the multiple matches to create a subgroup of matches, wherein performing the one or more actions is based on the subgroup of matches.

7. The method of claim 6, wherein each patient is associated with a value, and wherein the removing of the one or more outlier matches comprises:
detecting one or more patients having an associated value within a highest and/or lowest percentage among all associated values of the multiple patients; and
excluding the matches among the multiple matches that include any of the detected patients.

8. The method of claim 1, wherein the multiple patients are associated with one or more categories, and performing the one or more actions is based on the one or more categories.

9. The method of claim 1, further comprising receiving a plurality of values, each value associated with each patient among the multiple patients and being based on a cost for treatment.

10. The method of claim 9, wherein the performing of the one or more actions further comprises:
determining a distribution of the values, and
displaying, on a user interface, the determined distribution.

11. The method of claim 10, wherein determining the distribution of the values includes comparing, within each of the multiple matches, the value associated with the treatment patient and the value associated with the control patient.

12. The method of claim 10, wherein the assigning, sorting, matching, and performing is repeated a plurality of iterations to determine a plurality of distributions of the values, each distribution corresponding to an iteration, and the method further comprises:
determining, based on the plurality of distributions, a representative iteration among the plurality of iterations; and
displaying, on the user interface, a table indicating the representative iteration.

13. The method of claim 12, wherein determining the representative iteration is based on:
a number of the values of the distribution for each iteration that have a same sign as a mean of the distribution; and
a magnitude of a difference between the values of the distribution for each iteration and the mean.

14. The method of claim 13, further comprising determining a centroid based on the magnitudes and signs of the values for all iterations, wherein determining the representative iteration is based on a difference between each iteration and the determined centroid.

15. The method of claim 1, wherein the matching is based on one or more characteristics of the multiple patients.

16. A system for evaluating programs, the system comprising:
a memory having processor-readable instructions stored therein; and
a processor configured to access the memory and execute the processor-readable instructions to perform operations comprising:
determining a propensity score, using a propensity score model, for each patient among multiple patients, wherein the multiple patients include a plurality of treatment patients and a plurality of control patients, and the propensity score represents a probability of assignment to treatment;
assigning a random value to each patient in an assignment group, the assignment group including at least one of the plurality of treatment patients or the plurality of control patients;
sorting the plurality of patients based on the assigned random values;
matching, based on the sorted patients and the determined propensity scores, each treatment patient to a control patient to create multiple matches, each match including one treatment patient and at least one control patient; and
performing, based on the multiple matches, one or more actions related to the multiple patients.

17. The system of claim 16, wherein the operations further comprise repeating assigning the random value, sorting the plurality of patients, and matching, wherein, upon repeating assigning the random value, each patient in the assignment group is assigned a random value that is different from the random value previously assigned.

18. The system of claim 17, wherein the assigning, sorting, matching, and performing is repeated a plurality of iterations to determine a plurality of distributions of the values, each distribution corresponding to an iteration, and the method further comprises:
determining, based on the plurality of distributions, a representative iteration among the plurality of iterations; and
displaying, on the user interface, a table indicating the representative iteration.

19. A non-transitory computer-readable medium storing a set of instructions that, when executed by a processor, perform operations for evaluating programs, the operations comprising:
determining a propensity score, using a propensity score model, for each patient among multiple patients, wherein the multiple patients include a plurality of treatment patients and a plurality of control patients, and the propensity score represents a probability of assignment to treatment;
assigning a random value to each patient in an assignment group, the assignment group including at least one of the plurality of treatment patients or the plurality of control patients;
sorting the plurality of patients based on the assigned random values;
matching, based on the sorted patients and the determined propensity scores, each treatment patient to a control patient to create multiple matches, each match including one treatment patient and at least one control patient; and
performing, based on the multiple matches, one or more actions related to the multiple patients.

20. The computer-readable medium of claim 19, wherein the operations further comprise repeating assigning the random value, sorting the plurality of patients, and matching, wherein, upon repeating assigning the random value, each patient is assigned a random value that is different from the random value previously assigned.
